(19) United States
(12) Patent Application Publication
(10) Pub. No.: US 2009/0259093 A1
(43) Pub. Date: Oct. 15, 2009

(54) ARTIFICIAL SPHINCTER WITH PIEZOELECTRIC ACTUATOR

(76) Inventors: Nikhil D. Bhat, Fremont, CA (US); Anant V. Hegde, Hayward, CA (US); George Yoseung Choi, Menlo Park, CA (US); Wally S. Buch, Atherton, CA (US)

Correspondence Address: SHAY GLENN LLP, 2755 CAMPUS DRIVE, SUITE 210, SAN MATEO, CA, 94403, US

(21) Appl. No.: 12/420,744

(22) Filed: Apr. 8, 2009

Related U.S. Application Data
(60) Provisional application No. 61/044,885, filed on Apr. 14, 2008.

(51) Int. Cl. A61F 2/02

(52) U.S. Cl. 600/31

(57) ABSTRACT

A biologically implantable artificial sphincter system and methods of using the same is disclosed. The artificial sphincter system disclosed herein comprises a sphincter band and a piezoelectric element, both of which are adapted and configured for coordinated operation to open and/or close a body cavity. The artificial sphincter systems are useful in the treatment of urinary incontinence, fecal incontinence, and reflux disorders. The implanted artificial sphincter can also provide a signal to the recipient to urinate or defecate.

### CROSS REFERENCE TO RELATED APPLICATIONS

This application claims the benefit of U.S. Provisional Application No. 61/044,885 filed Apr. 14, 2008. This application is also related to U.S. Non-Provisional application Ser. No. 11/213,438 filed Aug. 25, 2005. Each of these applications is incorporated herein by reference in its entirety.

### FIELD OF THE INVENTION

This invention relates to artificial sphincters actuated by one or more piezoelectric motors adapted and configured to replace or augment natural sphincters, such as urinary, fecal and gastric sphincters, and methods of using the same.

### BACKGROUND OF THE INVENTION

It is estimated that over 12 million Americans have urinary incontinence. Incontinence affects all ages, both sexes, and people of every social and economic level. It is also estimated that 15 to 30 percent of people over the age of 60 have incontinence. Women are twice as likely as men to have this condition. In addition, at least half of the 1.5 million Americans who reside in nursing homes are incontinent. The exact number of people with incontinence is not known, but the total number of people affected may be far greater than current estimates. Incontinence is a symptom that can be caused by a wide variety of conditions. Some of these causes, such as urinary tract or vaginal infections, medicine effects, or constipation, may be temporary. In addition, to urinary incontinence, fecal incontinence and reflux diseases are common disorders caused by malfunctioning sphincters.

Artificial sphincters that are in the market today have several components such as a pump, fluid reservoir, cuff, one-way valves and the tubing that connects the reservoir, pump and the cuff. It is not very comfortable for the patient to use these systems. Erosion, fluid loss, pressure loss, etc. compromise the effectiveness of the artificial sphincters over-time. Hence, there is a need to develop novel sphincters for use in disorders caused by the malfunction of natural sphincters in the body.

### SUMMARY OF THE INVENTION

The invention provides an artificial sphincter and methods of use thereof. The artificial sphincter comprises a support i.e. sphincter band for the placement around a body cavity and a piezo actuator element i.e. in the form of a piezo motor for opening and closing of the sphincter band. The artificial sphincter can be used around several body cavities, including the urethra and various parts of the gastro-intestinal tract. The sphincter system allows for opening and/or closing of the body cavity around which it is placed, this opening and closing being controlled by the activation of the piezo actuator element with electrical signals i.e. using a driver circuit. The artificial sphincter system can also include a sensor to sense the state of the body cavity it surrounds to provide signals for activation or inactivation of the piezo actuator element.

An aspect of the invention is an artificial sphincter comprising a piezo actuator element and a sphincter band, both of which are configured to allow the constriction of a body cavity by the sphincter band driven by the piezo actuator element. The artificial sphincter is useful for constriction of various body cavities, including the urethra to treat urinary incontinence; the esophagus to treat reflux disease, and the intestine to treat fecal incontinence. In some embodiments, the artificial sphincter further comprises electrical terminals contacting the piezo actuator element for modulating the shape of the sphincter band. The sphincter band can be rigid or semi-rigid such as to provide a certain amount of stiffness for pinching the body cavity with the sphincter band.

In some embodiments, the artificial sphincter includes a control unit for electrically controlling the piezo actuator element to open or close the body cavity. The action of the artificial sphincter of the present invention can be controlled using a variety of control units, for example, (a) power source and a simple switch or (b) power source and a logic/control device such as a computer. The artificial sphincters of the present invention can also comprise a sensing system (such as a system comprising strain gauges) for sensing the degree of contraction of the sphincter band. Elements of the sensing system may be incorporated into or mounted on the sphincter band.

In one embodiment of the invention, the power source and the switch are implanted in the patient's body just beneath the outer skin. This embodiment may also include a battery recharging mechanism implanted in the patient's body. In another embodiment of the invention, the power source is outside the patient's body and the power is transmitted transcutaneously through the induction coil that is implanted in the patient's body. In another embodiment of the present invention, the actuator used is a superelastic shape memory alloy like Nitinol. In another embodiment of the invention, the actuator used is an electroactive polymer. In another embodiment of the invention, the actuator used is a conducting polymer.

In one embodiment, the invention comprises of a biologically implantable artificial sphincter comprising piezo motor element, a sphincter band, and a driver/power receiver circuit. The sphincter band can be made out of an elastomeric material like silicone, polyurethane, thermoplastic elastomers like Santoprene, Kratons, etc. or combination there of. The sphincter band can be reinforced with Kevlar fiber, nylon, carbon fiber, etc. to improve the durability or fatigue life.

In some embodiments, the piezo actuator element is made out of ceramic material. Alternatively, the actuator can be made out of polymeric materials like polyvinylidene fluoride.

Another aspect of the invention is a method of opening and/or closing a body cavity using an artificial sphincter described herein. For example, one embodiment is a method of treating urinary incontinence using an artificial sphincter comprising implanting the artificial sphincter around the urethra; closing the urethra with artificial sphincter; and opening the urethra by transmitting an electrical signal to the artificial sphincter; wherein opening the urethra comprises the electrical signal actuating a piezo actuator in the artificial sphincter. The artificial sphincters described herein can also be used for the treatment of fecal incontinence and reflux diseases.

The artificial sphincter of the present invention may be adapted for placement around a number of body lumens, including the urethra, the anal canal, and the lower esophagus. One aspect of the invention is an artificial sphincter having a piezoelectric element attached to a housing, a drive element movably coupled to the housing, a driver attached to the piezoelectric element and configured to translate a piezoelectric oscillation of the piezoelectric element into movement of the drive element and a sphincter band attached to the housing and configured to be driven by the drive element. In one alternative, the length of the sphincter band is configured to encircle a body cavity, organ or lumen. According to one aspect, the body cavity is a urethra, lower esophagus, lower gastro-intestinal tract, or rectum. In another alternative, the driver is configured to translate a piezoelectric oscillation of the piezoelectric element into longitudinal movement of the drive element. In another alternative, the driver is configured to translate a piezoelectric oscillation of the piezoelectric element into rotational movement of the drive element. According to one aspect, the artificial sphincter includes a power supply in electrical communication with the piezoelectric element. In one embodiment, application of electrical current to said piezoelectric element increases or decreases a circumference formed by the sphincter band. In one aspect, the perimeter formed by the sphincter band is adapted and configured to at least partially encircle a body cavity. In one embodiment, the sphincter includes a bellows between the drive element and the housing. In one embodiment, the sphincter includes an inductive coupling mechanism adapted to connect the piezoelectric element to a power source.

In another aspect of the invention, there is a method of controlling passage of contents across a body cavity including the steps of implanting a control device around a body cavity, the device comprising a piezoelectric actuator, a sphincter band, a drive element connected between the piezoelectric actuator and the sphincter band, and a power management device. Next, there is a step of controlling a flow of contents in said body cavity, said control being performed by constricting and unconstricting said body cavity by operating the piezoelectric actuator to increase and decrease the circumference of the sphincter band.

In another aspect of the invention, there is a method of treating a disease using an artificial sphincter including the steps of implanting a sphincter band around a body cavity, said sphincter band connected to a piezoelectric element and closing said body cavity with the sphincter band by converting the movement of the piezoelectric element to movement to decrease the diameter of the sphincter band.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B illustrate a male and female urinary system.

FIGS. 2A and 2B illustrate an embodiment of the artificial sphincter system in use in a female and a male urinary system.

FIG. 3A illustrates a section view of an embodiment of an artificial sphincter.

FIG. 3B illustrates a top down view of section A-A of the embodiment of FIG. 3A.

FIG. 3C illustrates an enlarged view of a portion of FIG. 3A.

FIG. 4 illustrates an enlarged view of a piezoelectric motor and its mode of operation.

FIG. 5 illustrates a cross-sectional view of the upper gastro-intestinal tract.

FIG. 6 illustrates an embodiment of the artificial sphincter system in use in upper gastro-intestinal tract.

FIG. 7 illustrates a cross-sectional view of the lower gastro-intestinal tract.

FIG. 8 illustrates a cross-sectional view of an embodiment of the artificial sphincter in use in a lower gastro-intestinal tract.

FIG. 9A illustrates an embodiment of an inductive coupling system associated with the artificial sphincter system.

FIG. 9B illustrates other aspects of an inductive coupling system associated with the artificial sphincter system.

FIGS. 10 and 11 illustrate an operational prototype of a piezoelectrically actuated artificial sphincter with the sphincter band in a closed configuration (FIG. 10) and an open configuration (FIG. 11).

FIG. 12A illustrates a piezoelectric housing, lead screw and communication cable of an operational prototype.

FIG. 12B illustrates a piezoelectric housing, lead screw and bellows assembly.

FIG. 12C is a cross-sectional view of the assembly shown in FIG. 12B.

FIG. 12D illustrates an alternative embodiment of a sealing capsule around the entire piezoelectric housing and lead screw assembly of an artificial sphincter.

FIG. 13 illustrates the driver circuit and associated electronics and various cables used in the operational prototype.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the claims define the scope of the invention and that methods and structures within the scope of these claims and their equivalents be covered thereby.

Embodiments of the invention provide artificial sphincters and methods of use thereof. The artificial sphincter comprises a sphincter band for placement around a body cavity or positioning adjacent to a natural sphincter. The sphincter band is attached to a housing containing a piezoelectric driver. The piezoelectric driver utilizes the movement of a piezoelectric element to engage and move a drive element. Movement of the drive element causes movement of the sphincter band to open and close (including the ability to partially open, or partially close) with respect to the body cavity.

An aspect of the invention is an artificial sphincter comprising a piezoelectric element coupled to a sphincter band that is configured to allow the constriction of a body cavity using the band moved by the piezoelectric element. The artificial sphincter is useful for constriction of various body cavities, including the urethra to treat urinary incontinence; the esophagus to treat reflux disease, and the rectum to treat fecal incontinence. The piezoelectric element also includes suitable electrical terminals connecting the piezoelectric element to a power source, control electronics, driver circuits and the like. The sphincter band can be rigid or semi-rigid such as to provide a certain amount of stiffness for constricting the body cavity when moved by the piezoelectric element.

The artificial sphincter comprises a sphincter band adapted and configured for placement around a body cavity. The length, width, shape and other physical properties of the sphincter band may be adjusted based on a number of factors such as the body cavity to be treated as well as the characteristics and mechanical operation of the piezoelectric element, the piezoelectric driver and the drive element. In one aspect, the sphincter band is sufficiently long to at least partially encircle the body cavity to be treated. The sphincter band can also include a soft elastomeric layer to protect the body lumen or cavity in use.

In one embodiment of the invention, the power source and the switch are implanted in the patient's body just beneath the outer skin. This embodiment may also include a battery recharging mechanism implanted in the patient's body. In another embodiment of the invention, the power source is outside the patient's body and the power is transmitted transcutaneously through the induction coil that is implanted in the patient's body. In another embodiment of the present invention, the actuator used is a superelastic shape memory alloy like Nitinol. Preferably, the sphincter also includes a power supply in electrical communication with the piezoelectric element and a switch.

Another aspect of the invention is a method of opening and/or closing a body cavity using an artificial sphincter described herein. For example, one embodiment is a method of treating urinary incontinence using an artificial sphincter comprising implanting the artificial sphincter around the urethra; closing the urethra as a result of piezoelectric movement of a piezoelectric element in the artificial sphincter; and opening the urethra by transmitting an electrical signal to the artificial sphincter; wherein opening the urethra comprises the electrical signal actuating an piezoelectric element in the artificial sphincter to open. The artificial sphincters described herein may also be used for the treatment of fecal incontinence and reflux diseases.

The artificial sphincter bands of the present invention may be adapted for placement around a number of body lumens, including the urethra, the anal canal, and the lower esophagus.

Another embodiment of the invention is an implantable control device comprising a piezoelectric actuator, a band, and a power management device; wherein the band is configured to encompass a body cavity, the piezoelectric actuator and the band are configured to constrict the body cavity, and the power management device is adapted to connect to the piezoelectric actuator.

The devices disclosed herein can be coated with materials to prevent or promote tissue growth. Also, the devices can include an inductive coupling mechanism adapted to connect the piezoelectric element to a power source. The body cavities regulated by the artificial sphincters disclosed herein include urethra, lower esophagus, lower gastro-intestinal tract, or rectum.

Another embodiment of the invention is a method of controlling passage of contents across a body cavity comprising implanting a device around a body cavity, the device comprising an piezoelectric actuator, a band, and a power management device; controlling a flow of contents in the body cavity, this control being performed by constricting and unconstricting of the body cavity with the band under forces generated by the piezoelectric actuator. In this method control of flow of contents in the body cavity can be in response to transcutaneous feedback from the body cavity, said feedback being related to the contents in the body cavity. The control device described herein can be controlled with an inductive coupling mechanism. The inductive coupling mechanism can be transcutaneous.

The devices described herein are suitable for the treatment of several disorders such as disorders of the urethra, lower esophagus, lower gastro-intestinal tract, or rectum. One embodiment is a method of treating a disease using an artificial sphincter comprising implanting a band of an artificial sphincter around a body cavity, the artificial sphincter comprising an piezoelectric element and a band; closing the body cavity with the artificial sphincter by applying a mechanical force on the body cavity using the band under movement from the piezoelectric element; and opening the body cavity by transmitting an electrical signal to the piezoelectric element to move the band to open the body cavity. This method may be used in the treatment of urinary incontinence, fecal incontinence, or reflux diseases.

### Artificial Sphincter System

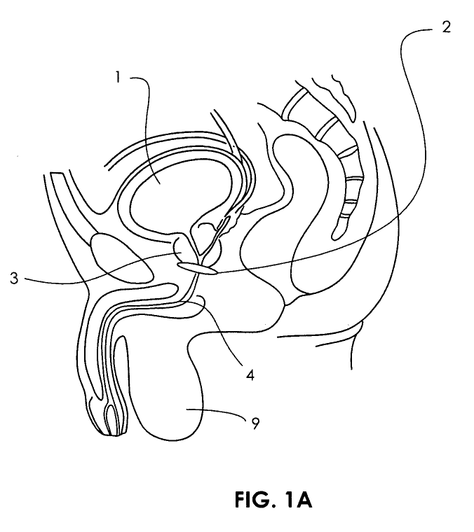

FIGS. 1A and 1B depict the male and female urinary system. Some of the components of a male urinary system, as depicted in FIG. 1A, are the urinary bladder 1, prostate gland 3, urinary sphincter muscle 2, urethra 4, and scrotum 9. The components of a female urinary system, as depicted in FIG. 1B, are the urinary bladder 1, uterus 8, urinary sphincter muscle 2, and urethra 4.

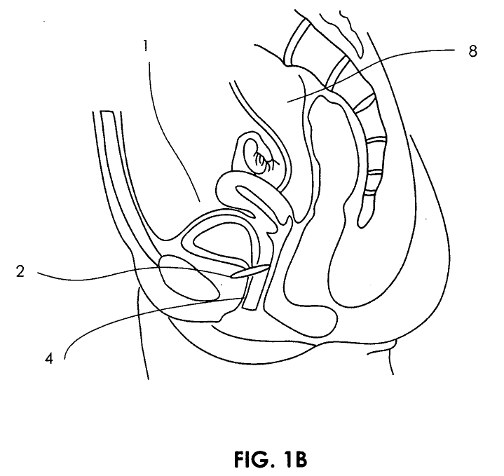

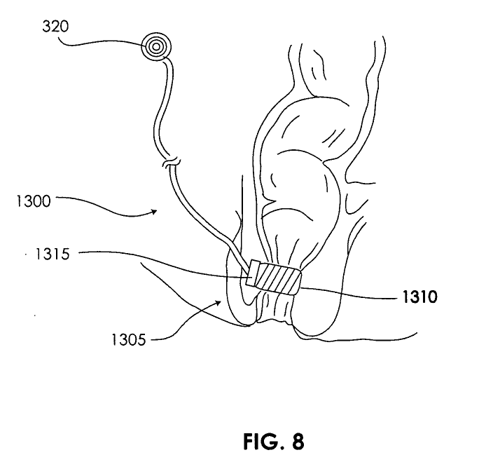

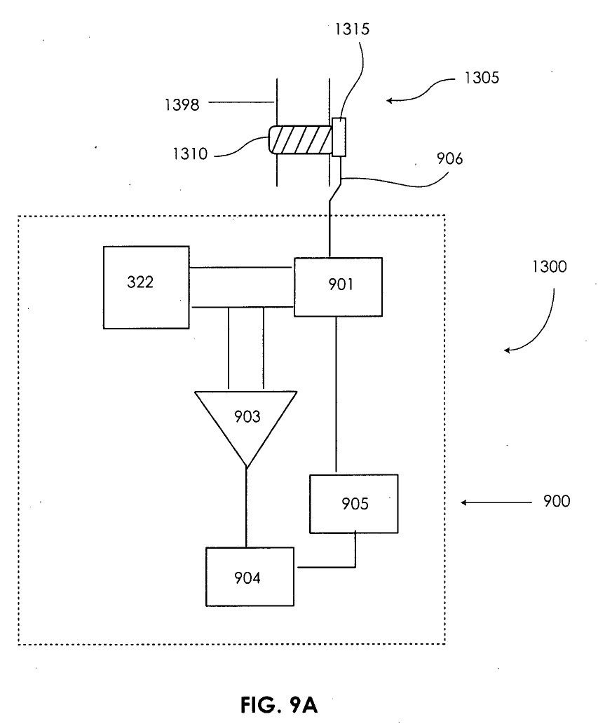

FIG. 2A illustrates an embodiment of an artificial sphincter system 1300 implanted in a female subject. The artificial sphincter system 1300 discussed herein comprises an artificial sphincter 1305 and an inductive coupling system 900 such as depicted in FIG. 9A. The artificial sphincter system in the female subject is similar to the artificial sphincter system shown for the male subject in FIG. 2B. In the illustrated embodiment, in FIGS. 2A and 2B, the artificial sphincter 305 is controlled with a switch 320 and a power source 322. The switch and power source may be located inside or outside the body. In this view, the sphincter band 1310 and the housing 1315 are visible. The sphincter band 1310 is extending about the urethra 4.

FIG. 2B illustrates an embodiment of an artificial sphincter system 1300 implanted in a male subject. The subject has a bladder 1, a sphincter muscle 2, a prostate gland 3 and a urethra 4. As depicted, the artificial sphincter system 300 has an artificial sphincter 305, a switch 320, and a power source 322. The switch 320 and/or power source 322 can be connected to the artificial sphincter 305. In this view, the sphincter band 1310 and the housing 1315 are visible. The sphincter band 1310 is extending about the urethra 4.

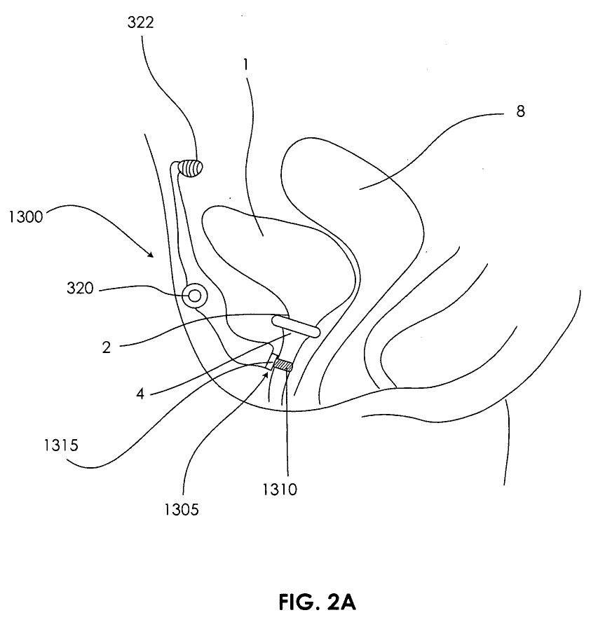

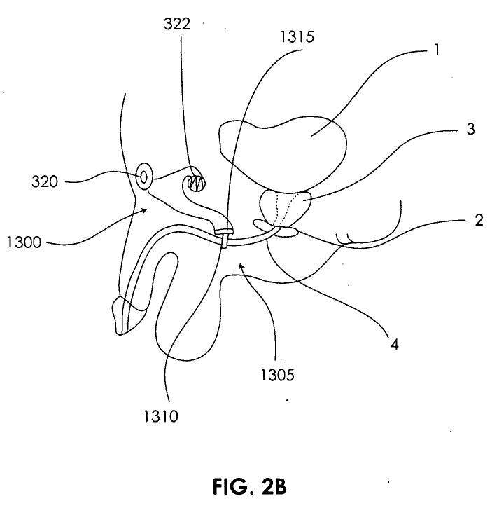

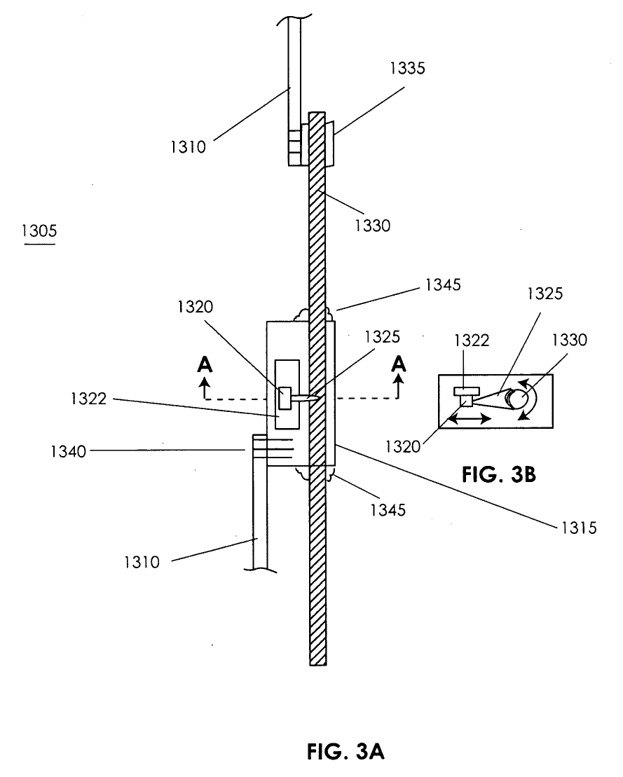

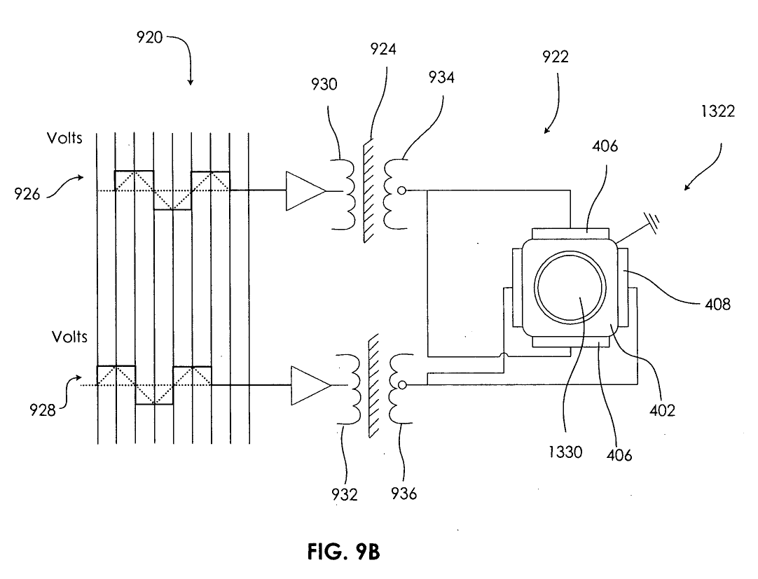

FIGS. 3A-3C illustrate one specific embodiment of a piezoelectrically actuated artificial sphincter 1305. FIG. 3A is a section view through a piezomotor housing 1315 containing a piezomotor 1322 and a piezoelectric element 1320. The piezoelectric element 1320 is attached to a piezoelectric driver 1325 that is in turn connected to a drive element 1330. The drive element 1330 is attached at one end to the sphincter band 1310. The other end of the sphincter band 1310 is attached to the piezomotor housing 1315. The piezomotor housing is anchored (as appropriate to the circumstances and surrounding anatomy) within the body to allow the sphincter band 1310 to operate to constrict or open about the body lumen as desired. In the embodiment of FIG. 3A the drive element 1330 is a lead screw with threads. Lead screw seals 1345 are provided at both ends of housing 1315 to seal the internal components within the housing from bodily fluids which may damage them. The piezoelectric driver 1325 is a v-shaped member attached at one end to the piezoelectric element 1320. The other end of the piezoelectric driver 1325 is shaped to engage with the threads. Movement of the piezoelectric driver 1325 will cause the lead screw to rotate. The piezoelectric element 1320 may be driven by signals and power from the driver circuit/electronics module to produce bi-directional lead screw movement. One direction of lead screw movement will shorten the sphincter band circumference and constrict the body lumen (i.e., move the lead screw attachment end 1335 towards the piezomotor housing, see FIG. 10). The other direction of movement will lengthen the sphincter band circumference and lessen the constriction on the body lumen (i.e., move the lead screw attachment end 1335 away from the piezomotor housing, see FIG. 11). Mechanical stops (not shown) may be provided to physically limit the amount movement that the lead screw can make in one or both directions. These stops may be configured to be adjustable such that they can be set by a surgeon when the device is being implanted and tested.

FIG. 3B is a top down view of section A-A in FIG. 3A. The movement of the piezoelectric element 1320 (indicated by arrows) produces movement on one end of the piezoelectric driver 1325. The other end of the piezoelectric driver 1325 is configured, in this embodiment, to engage with the threads of the lead screw, as best seen in FIG. 3C. As such, the movement of the piezodriver 1325 produces rotational movement of the lead screw 1330. The advancement of lead screw 1330 produces opening or closing of the sphincter band circumference. The piezoelectric element 1320 may be operated to produce bi-directional rotation (as indicated by the arrow) of the lead screw in any amount desired to produce the desired sphincter band movement.

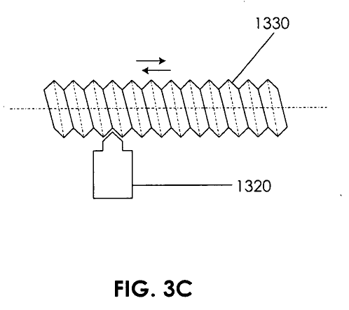

FIG. 4 illustrates the mode of operation of exemplary piezomotor 1322 described above. Piezomotor 1322 has an elongate housing 1315 with a nut 402 secured within one end and a bushing 404 secured in the other end. Lead screw 1330 is received through housing 1315, is threadably engaged by nut 402, and slideably engaged by busing 404. A first pair of piezo crystal elements 406, 406 are located along opposite sides of housing 1315. A second pair of piezo crystal elements 408, 408 (only one shown in FIG. 4) are located along the other opposite sides of housing 1315, orthogonal to the first pair of elements 406, 406.

In operation, one pair of piezo crystal elements is actuated at a time. One element of the pair is energized to contract, while the other element is energized to expand or is not energized. This causes nut 402 to move laterally towards the side of housing 1315 having the piezo crystal element that is contracting. By sequentially energizing each of the elements to contract one at time in one direction around housing 1315, nut 402 is made to orbit or wobble around lead screw 1330 in a “Hula Hoop” manner. This motion of nut 402 in turn causes lead screw 1330 to turn in that same direction, and move axially as previously described. By reversing the order in which the piezo crystal elements are energized, the direction of rotation of nut 402 and lead screw 1330 is reversed, causing lead screw 1330 to move axially in the opposite direction. The dotted lines in FIG. 4 depict the motor vibration mode shape caused by the opposing strain of the piezo crystal elements, with nodes shown by reference numerals 410.

Piezomotor 1322 can be configured to generate no magnetic fields and can be constructed entirely of non-ferromagnetic materials. No coils, magnets or iron cores are needed. In some embodiments, the motors use nonmagnetic stainless steel, bronze and/or titanium. Non-magnetic motors are safe for use in Magnetic Resonance Imaging (MRI) systems because they are not attracted by high magnetic fields. In addition, these motors have demonstrated compatibility with the MRI imaging process when stationary and moving.

Further details of piezomotor construction and operation may be found in U.S. Pat. No. 6,940,209 issued Sep. 6, 2005, to David A. Henderson, in an article authored by David A. Henderson entitled Simple Ceramic Motor . . . Inspiring Smaller Products, Actuator 2006, 10^{th} International Conference on New Actuators, 14-16 Jun. 2006, Bremen, Germany, and at www.newscaletech.com.

The piezoeletrically actuated artificial sphincter may be used around several body cavities, including the urethra and various parts of the gastro-intestinal tract. The sphincter system allows for opening and/or closing of the body cavity around which it is placed, this opening and closing being controlled by the activation of the piezoelectric element with electrical signals. The artificial sphincter system can also include a sensor to sense the state of the body cavity it surrounds to provide signals for activation or inactivation of the piezoelectric element.

An elastomeric layer may be provided on the sphincter band or other components of the artificial sphincter to improve biocompatibility. The elastomeric layer can be made of or coated with silicone, latex, polychloroprene (e.g., neoprene), fully vulcanized thermoplastic rubbers (TPRs) such as polyolefin-based or styrene-based rubbers (e.g., Alcryn® from Dupont, Krytont from Shell, Santoprene® from Monsanto), thermoplastic elastomers (TPEs) such as polyester TPEs or nylon TPEs (e.g., Hytel® from Dupont, Lomod® from GE, Pebax® from Elf AtoChem), Teflon® from Dupont, Gore-Tex from W. L. Gore & Associates, Inc., polypropylene or any other thermoplastic or thermosetting plastic polymer. One or more of these materials may be used in sheet form, as a mesh, a coating, or otherwise interposed between the body of the sphincter band and the tissue it surrounds to inhibit tissue erosion.

In some embodiments, the artificial sphincter is controlled with a transcutaneous energy transmission system (TETS) and/or a processor. The TETS transmitter coil may be located outside the body. The processor is configured to control the artificial sphincter and also sense and/or process other relevant information necessary for control of the body cavity, such as the urethra.

In some embodiments, a power supply 322 (e.g., a battery) and an ON/OFF switch 320 are implanted in the subject. The power supply 322 and the switch 320 can be anywhere in the subject that is convenient to the subject. Wires connect the power supply 322, the switch 320 and the artificial sphincter 305. In other embodiments, the power supply 322 is outside the body of the subject. In such embodiments power can be transmitted to the artificial sphincter 305 using a transcutaneous energy transfer system (TETS), for example a system that inductively transmits energy (i.e., similar to methods for delivering power to artificial hearts). In other embodiments, power supplies may be located both internal and external to the body, and power, control signals, and/or feedback signals may be inductively transmitted through the skin.

FIG. 9A depicts one exemplary inductive coupling system 900 that is suitable for controlling the artificial sphincter 1305. The sphincter band 1310 and housing 1315 are shown about a body lumen 1398. A connecting element 906 (which connects the electrical contacts 310 (not shown in this view) within the housing 1315 to the rest of the electrical system), a connector 901, an energy source 322, a sensor 903, a timer 904, and a controller 905. The connector 901, energy source 322, sensor 903, a timer 904, and controller 905 may be located in a housing disposed in a region outside or inside the body.

The energy source or power supply 322 can be a power cell, a battery, a capacitor, a substantially infinite bus, a portable generator, or combinations thereof. The power supply typically has a power output of from about 1 mA to about 5 A. The connecting element 906 may be a wire lead, an inductive energy transfer system, a conductive energy transfer system, a chemical transfer system, an acoustic or otherwise vibratory energy transfer system, a nerve or nerve pathway, other biological tissue, or combinations thereof. The connecting element is made from one or more conductive materials, such as copper. The connecting element is completely or partially insulated and/or protected by an insulator, for example polytetrafluoroethylene (PTFE). The insulator is typically biocompatible. The power supply 322 is in electrical communication with the piezoelectric element through a connecting element. The connecting element is attached to the electrical contacts 310.

The artificial sphincter can be controlled with a sensor and a controller to open and close the body cavity, such as the urethra. The controller can be a programmable device to open and close the body cavity via the sphincter band as moved by the operation of the piezoelectric element. The sensor can be a pressure sensing device that can sense the pressure in the body cavity, such as urinary bladder or gastro-intestinal tract, and send a signal to the controller.

FIG. 9B provides further details of an exemplary inductive coupling system that may be used to operate the artificial sphincter described above. The system includes an external power transfer system 920 and an implanted system 922 separated from each other by the patient's skin 924. External system 920 provides two separate motor drive signals 926 and 928 that are 90 degrees out of phase from one another. When drive signal 926 is 90 degrees advanced from drive signal 928, piezomotor 1322 is driven in one direction. When drive signal 926 is 90 degrees retarded from drive signal 928, piezomotor 1322 is driven in the opposite direction.

External power transfer system 920 further includes two transmitter coils 930 and 932. Drive signal 926 is fed into transmitter coil 930 and drive signal 928 is fed into transmitter coil 932. External system 920 may be located within a handheld housing, with transmitter coils 930 and 932 located near an exposed surface of the housing at a predetermined spacing. Receiver coils 934 and 936 may be mounted beneath the skin of the patient with the same spacing. In operation, the housing is positioned over the patient's skin such that transmitter coil 930 is generally aligned with receiver coil 934 and transmitter coil 932 is generally aligned with receiver coil 936. Drive signals 926 and 928 may then be transferred by induction through the skin 924 from the external system 920 to the implanted system 922. Drive signal 926 is transmitted from transmitter coil 930, through receiver coil 934 to the first pair of piezo crystal elements 406, 406 on opposite sides of piezomotor 1322. At the same time, drive signal 928 is transmitted from transmitter coil 932, through receiver coil 936 to the second pair of piezo crystal elements 408, 408 on opposite sides of piezomotor 1322. As described above in reference to FIG. 4, these drive signals cause piezomotor 1322 to advance lead screw 1330 in a desired direction. To drive piezomotor 1322 in the opposite direction, external system 920 may be commanded, such as with a pushbutton, to change the 90 degree phase shift between drive signals 926 and 928 to be in the opposite direction. Alternatively, the housing of external system 920 may be physically rotated 180 degrees so that transmitter coil 930 is generally aligned with receiver coil 936 and transmitter coil 932 is generally aligned with received coil 934 (i.e. opposite the configuration shown in FIG. 9B) to drive piezomotor 1322 in the opposite direction.

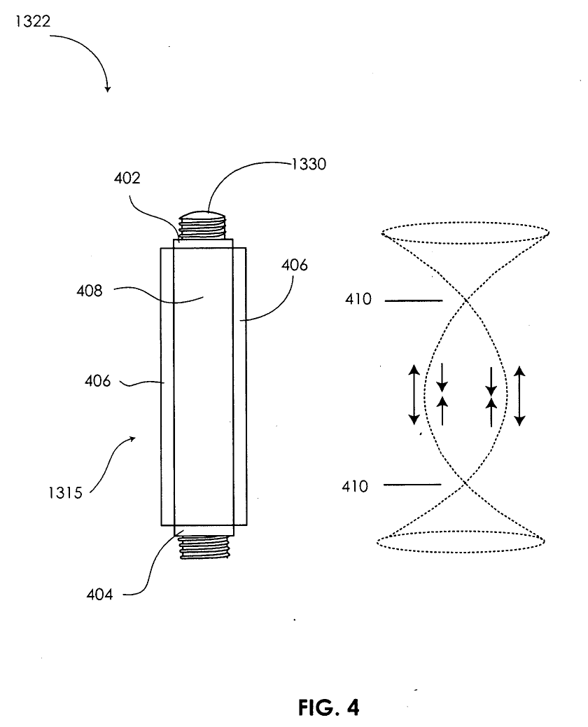

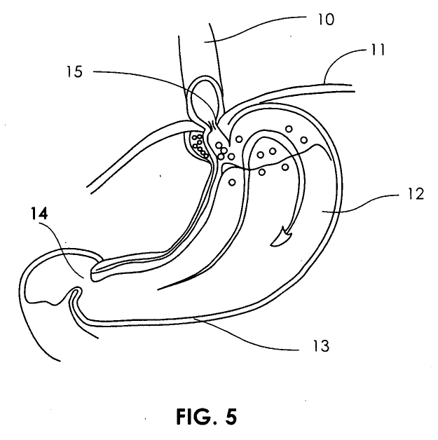

FIG. 5 depicts the upper gastro-intestinal tract with the esophagus 10, lower esophagus sphincter 15, diaphragm 11, stomach 13, liquid contents 12, and pylorus 14. FIG. 6 depicts the use of an artificial sphincter system 1300 in the esophagus for the treatment of reflux disorders with the artificial sphincter 1305 and switch 320. In this view, the sphincter band 1310 and the housing 1315 are visible. The sphincter band 1310 is extending about the esophagus 10.

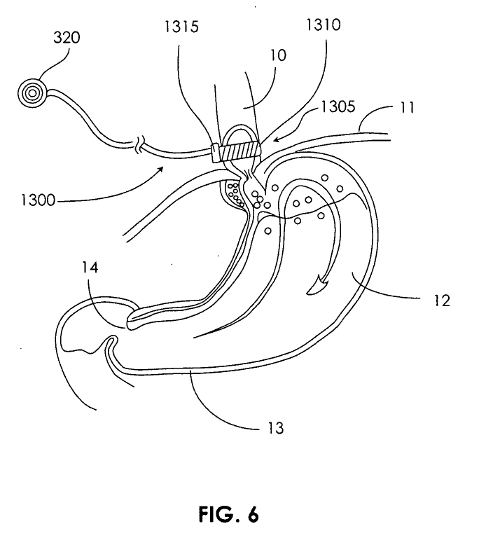

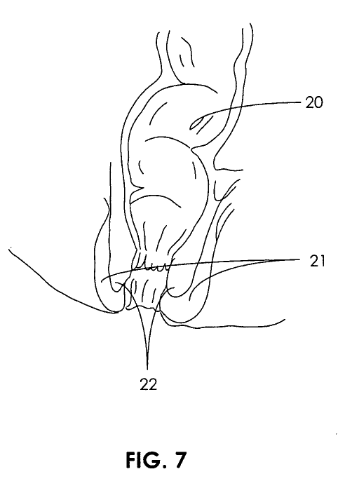

FIG. 7 depicts the lower gastro-intestinal tract with the rectum 20, exterior sphincter 21, and interior sphincter 22. FIG. 8 depicts the use of an artificial sphincter system 1300 in the rectum for the treatment of fecal incontinence with the artificial sphincter 1305 and switch 320. In this view, the sphincter band 1310 and the housing 1315 are visible. The sphincter band 1310 is extending about a portion of the lower gastro-intestinal tract.

The devices described herein maybe implanted with or without sutures or other bonding material such as surgical glue. The devices in some embodiments have external fibers or surface pores or coatings, such as protein based coatings like poly-L-lysine and poly-D-lysine, to promote tissue in-growth and help affix the device to adjacent tissue. In other embodiments, the devices are coated with material to prevent tissue growth around the implanted device, such as hyaluronic acid. This is particularly important in the prevention of tissue growth that may impede the operation of the piezoelectric element.

### Treatment of Diseases

The artificial sphincter systems disclosed herein are suitable for treatment of several diseases. These diseases include diseases caused by the malfunctioning of a body cavity and the resultant effects on the contents of the body cavity. Such diseases are typically caused due to the malfunctioning of sphincters and or valves that control these body cavities and/or due to the malfunctioning of peristaltic activity of the body cavity. Typically, these body cavities are tubular organs, such as the urethra, the gastro-intestinal tract, and blood vessels. The sphincter band of the artificial sphincters described herein is placed around a body cavity, such as a urethra, gastro-intestinal tract, and blood vessels. The diseases that can be treated include urinary incontinence, fecal incontinence, and reflux disorders. These sphincters are used by themselves, in combination with the natural sphincter and/or are used in combination with conventional therapies, including drugs, dietary modifications, and/or surgery. The artificial sphincters are also suitable for prophylactic uses.

### Urinary Incontinence

Urine is waste and water removed from the blood by the kidneys. Urine flows from the kidneys downward through a pair of tubes (the ureters) to the bladder. The bladder is a balloon-like container that stores urine. Urine leaves the body through another tube (the urethra) at the bottom of the bladder.

Urination is controlled by muscles, called sphincters, located at the base of the bladder and in the wall of the urethra. These normally stop the flow of urine. Usually, the sphincters close off the neck of the bladder and the urethra—like a tie around the bottom of a balloon—so that urine does not leak. When sphincters relax, they open the passage for urine. At the same time, the muscle of the bladder wall contracts (squeezes) and forces the urine out of the bladder. When urination is finished, the sphincters contract, and the bladder itself stops squeezing and relaxes.

Urinary incontinence is the medical term used to describe the condition whereby patient cannot control the flow of urine from the body. It usually happens because the sphincter is damaged. A damaged sphincter can not squeeze and close off the urethra. This means urine can leak or flow freely from the bladder. Many things can prevent the sphincter and bladder from doing their jobs. Most frequently, incontinence occurs in men when the sphincter and its nerves are affected by total or partial removal of the prostate to treat prostate cancer or other conditions. Sometimes an oversensitive or small bladder can put too much pressure on an otherwise healthy sphincter. Some other conditions include: urinary tract or vaginal infections, effects of medicine, constipation, weakness of certain muscles, blocked urethra due to an enlarged prostate, diseases and disorders involving nerves and/or muscles, and some types of surgery. Other causes can be longer-lasting, even permanent. These include such conditions as an overactive bladder muscle, weakness of the muscles holding the bladder in place, weakness of the sphincter muscles surrounding the urethra, birth defects, spinal cord injuries, surgery, or diseases involving the nerves and/or muscles (multiple sclerosis, muscular dystrophy, polio, and stroke). In some cases, more than one factor causes incontinence in a single individual.

Many types of treatment are available for incontinence depending on the type of incontinence one has. If the incontinence is due to the weakness of the sphincter muscle, artificial sphincter can be implanted to aid or replace the sphincter muscle.

The artificial sphincter disclosed herein can be used to replace or augment the patient's natural sphincters and when the patient feels the need to pass urine; the patient may activate the artificial sphincter by simply applying the power to the sphincter actuator transcutaneously. The sphincter can be used alone or in combination with other conventional treatments for urinary incontinence.

### Fecal Incontinence

Fecal incontinence is the inability to control the bowels. When someone with fecal incontinence feels the urge to have a bowel movement, they may not be able to hold it until they can get to a toilet or stool may leak from the rectum unexpectedly.

Fecal incontinence can have several causes including, but not limited to, constipation, damage to the anal sphincter muscles, damage to the nerves of the anal sphincter muscles or the rectum, loss of storage capacity in the rectum, diarrhea, and pelvic floor dysfunction. Fecal incontinence can be caused by injury to one or both of the ring-like muscles at the end of the rectum called the anal internal and/or external sphincters. The sphincters keep stool inside. When damaged, the muscles aren't strong enough to do their job, and stool can leak out. In women, the damage often happens when giving birth. The risk of injury is greatest if the doctor uses forceps to help deliver the baby or does an episiotomy, which is a cut in the vaginal area to prevent it from tearing during birth. Hemorrhoid surgery can damage the sphincters as well.

Treatment depends on the cause and severity of fecal incontinence; it may include dietary changes, medication, bowel training, or surgery. More than one treatment may be necessary for successful control since continence is a complicated chain of events. Food affects the consistency of stool and how quickly it passes through the digestive system. If a patient's stool is hard to control because it is watery, eating high fiber foods adds bulk and may make the stool easier to control. But people with well-formed stools may find that high fiber food acts as a laxative and contributes to the problem. Other food that may make the problem worse are drinks containing caffeine, like coffee, tea, and chocolate, which relax the internal anal sphincter muscle. If diarrhea is causing the incontinence, medication may help. Sometimes doctors recommend using bulk laxatives to help people develop a more regular bowel pattern. Or the doctor may prescribe antidiarrheal medicines such as loperamide or diphenoxylate to slow down the bowel and help control the problem. Bowel training helps some people re-learn how to control their bowels. In some cases, it involves strengthening muscles; in others, it means training the bowels to empty at a specific time of the day. Surgery may be an option for people whose fecal incontinence is caused by injury to the pelvic floor, anal canal, or anal sphincter. Various procedures can be done, from simple ones like repairing damaged areas, to complex ones like attaching an artificial anal sphincter or replacing anal muscle with muscle from the leg or forearm. People who have severe fecal incontinence that do not respond to other treatments may decide to have a colostomy, which involves removing a portion of the bowel. The remaining part is then either attached to the anus if it still works properly, or to a hole in the abdomen called a stoma, through which stool leaves the body and is collected in a pouch.

The artificial sphincter disclosed herein can be used to replace the patient's natural sphincters When the patient feels the need to have a bowel movement, the patient may activate the sphincter by simply applying power to the sphincter actuator transcutaneously. The artificial sphincter can be used alone or in combination with other conventional treatments for fecal incontinence.

### Reflux Disorders

Gastroesophageal reflux disease, commonly known as GERD or acid reflux. It is a condition in which the liquid content of the stomach regurgitates (backs up or refluxes) into the esophagus. The liquid can inflame and damage the lining of the esophagus and cause esophageal inflammation and damage (esophagitis).

The body has ways (mechanisms) to protect itself from the harmful effects of reflux and acid. For example, most reflux occurs during the day when individuals are upright. In the upright position, the refluxed liquid is more likely to flow back down into the stomach due to the effect of gravity. In addition, while individuals are awake, they repeatedly swallow, whether or not there is reflux. Each swallow carries any refluxed liquid back into the stomach. The salivary glands in the mouth produce saliva, which contains bicarbonate. The bicarbonate neutralizes the acid that remains in the esophagus. However, at night while sleeping, gravity is not in effect, swallowing stops, and the secretion of saliva is reduced. Therefore, reflux that occurs at night is more likely to result in acid remaining in the esophagus longer and causing greater damage to the esophagus.

The major factors that contribute to GERD involve the lower esophageal sphincter (LES), hiatal hernias (bulging of the esophagus between diaphragm and LES), esophageal contractions, and emptying of the stomach. The action of the lower esophageal sphincter (LES) is perhaps the most important factor (mechanism) for preventing reflux. Several different abnormalities of the LES have been found in patients with GERD. Two of them involve the function of the LES. The first is abnormally weak contraction of the LES, which reduces its ability to prevent reflux. The second is abnormal relaxations of the LES, called transient LES relaxations. They are abnormal in that they do not accompany swallows and they last for a long time, up to several minutes. These prolonged relaxations allow reflux to occur more easily. The transient LES relaxations occur in patients with GERD most commonly after meals when the stomach is distended with food. Transient LES relaxations also occur in individuals without GERD, but they are infrequent. The symptoms of uncomplicated GERD are primarily heartburn, regurgitation, and nausea. Some of the complications are ulcer, inflammation of the throat and larynx, and esophageal cancer.

Treatment for GERD includes life-style changes such as eating food at particular times of the day, not eating just before bed-time, eating food with less oil content, avoiding fried food, eating less spicy food, etc. Drugs that are used include antacids, such as Tums; histamine antagonists such as cimetidine (Tagamet), ranitidine (Zantac), nizatidine (Axid), and famotidine (Pepcid); proton pump inhibitors (PPI) such as omeprazole (Prilosec), lansoprazole (Prevacid), rabeprazole (Aciphex), pantoprazole (Protonix), and esomeprazole (Nexium); pro-motility such as metoclopramide (Reglan); and foam barriers such as the combination of aluminum hydroxide gel, magnesium trisilicate, and alginate (Gaviscon).

Treatment options include surgery. One of the procedures that is done to prevent reflux is technically known as fundoplication and is called reflux surgery or anti-reflux surgery. During fundoplication, any hiatal hernial sac is pulled below the diaphragm and stitched there. In addition, the opening in the diaphragm through which the esophagus passes is tightened around the esophagus. Finally, the upper part of the stomach next to the opening of the esophagus into the stomach is wrapped around the lower esophagus to make an artificial lower esophageal sphincter.

The artificial sphincter described herein may be used in combination with the conventional treatments for GERD, such as those listed herein. In some embodiments, the artificial sphincter is implanted above the LES around the esophagus and the induction coil is placed in the abdominal wall for powering the implant. The patient wears a power source and a transmitter coil similar to the one that is implanted in the abdominal wall. A microprocessor that may be embedded in either of the coils senses the activities of swallowing, coughing, etc. and controls the sphincter opening and closing events as needed.

FIGS. 10 and 11 are drawings of an operational prototype of a piezoelectrically actuated artificial sphincter with the sphincter band in a closed configuration (FIG. 10) and an open configuration (FIG. 11). The spacing (L) between the housing and the connection to the sphincter band is shorter in the closed configuration (Lc in FIG. 10) than in the open configuration (Lo in FIG. 11). As a result, the perimeter or circumference of the sphincter band is less in the closed configuration than in the open configuration. Thus, when in the closed configuration the body cavity undergoing treatment will be closed. When in the open configurations, the body cavity undergoing treatment is open. Partially open and partially closed configurations are also possible, if desired for a particular therapeutic outcome.

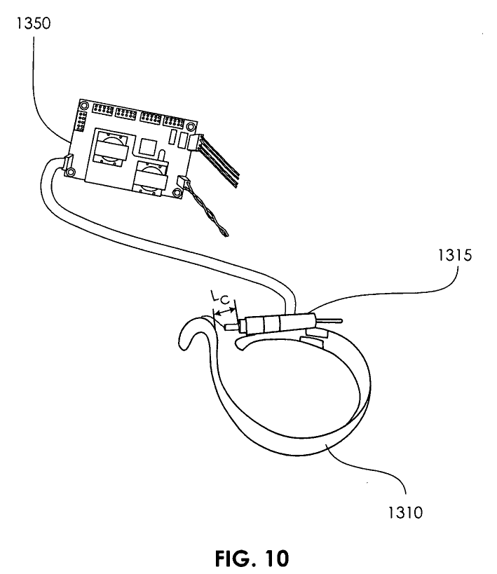

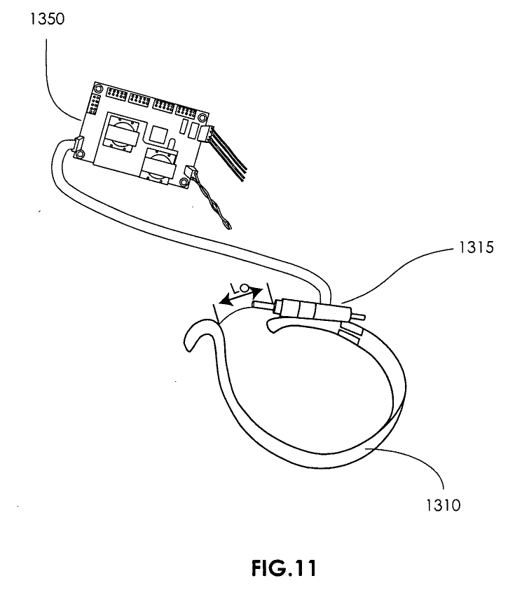

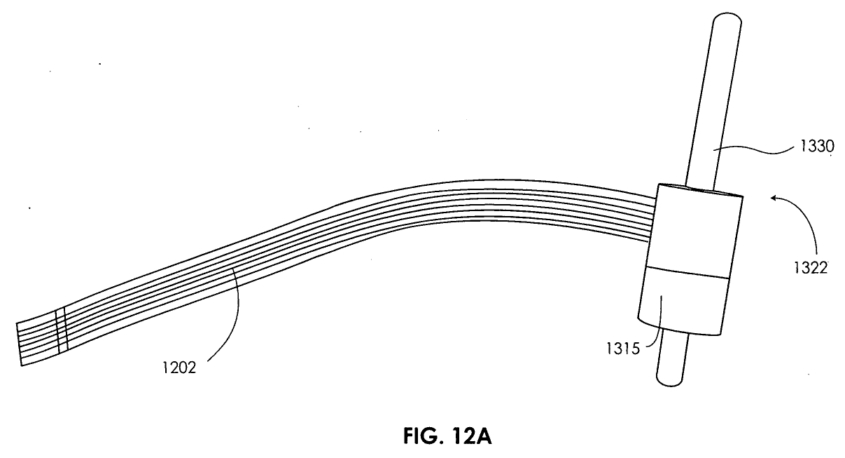

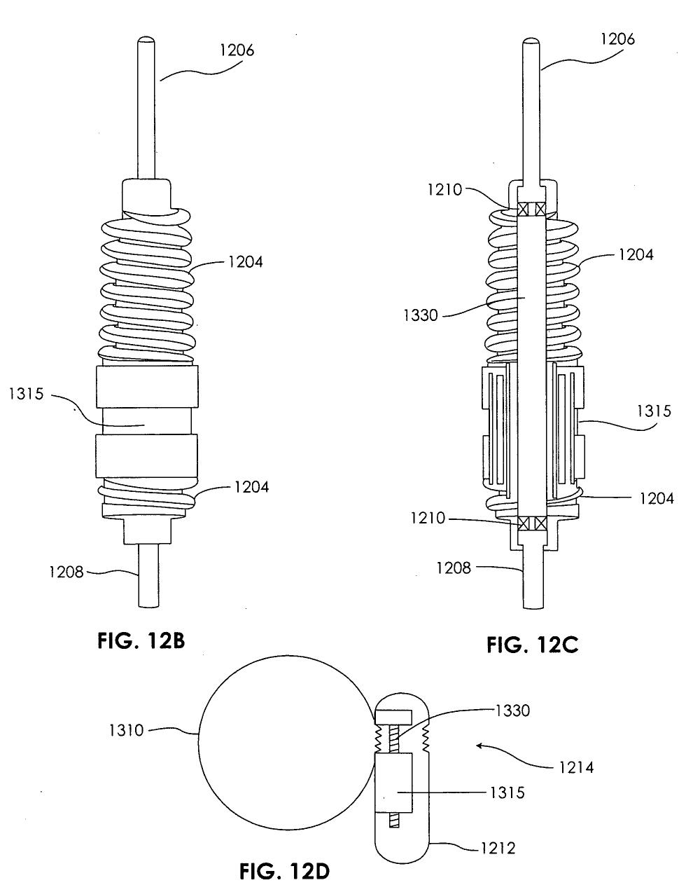

FIG. 12A is a drawing of an exemplary piezoelectric housing 1315, lead screw 1330 and communication cable 1202 of an operational prototype. FIGS. 12B and 12C show a side view and cross-sectional view of an alternative motor sealing arrangement. In this embodiment, a flexible bellows 1204 is located at each end of piezoelectric housing 1315. Non-rotating drive shafts 1206 and 1208 are located at each end of lead screw 1330, and are coupled to lead screw 1315 with bearings 1210. One end of each bellows 1204 seals against housing 1315 while the other end seals against its respective non-rotating shaft 1206 or 1208. As lead screw 1330 rotates and moves axially, each bellows 1204 either expands or contracts. In this manner, bellows 1204, 1204 serve to keep bodily fluids from coming in contact with lead screw 1330 and the interior of piezoelectric housing 1315. Bellows 1204 may be formed in a helical shape as shown to aid in manufacturability. In some embodiments, bellows 1204 is formed from polypropylene having a pore size of less than 15 microns to prevent tissue ingrowth after implantation.

FIG. 12D shows another arrangement for sealing piezoelectric housing 1315. In this embodiment, an expandable capsule 1212 surrounds the entire piezoelectric housing, lead screw and band mounting assembly. Capsule 1212 may be provided with a pleated bellows section 1214 as shown to accommodate movement of lead screw 1330 and sphincter band 1310. Alternatively, capsule 1212 may be oversized or configured to stretch. A power source, sensing, control and/or transmission electronics (not shown in FIG. 12D) may also be located in capsule 1212. In other embodiments (not shown), the entire artificial sphincter system including sphincter band 1310 may be enclosed within a toroid-shaped capsule that surrounds the body cavity like an inner tube.

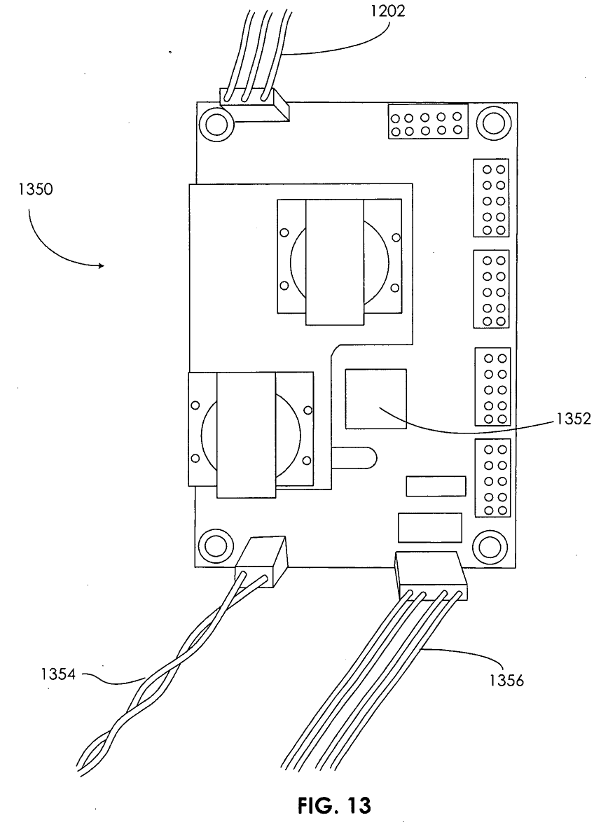

FIG. 13 is a drawing of the driver circuit and associated electronics and various cables used in the operational prototype. Circuit board 1350 includes a processor 1352 and connectors for receiving power supply cable 1354, communications cable 1202 to piezomotor 1322 (not shown), and communications cable 1356 to the external controller (not shown).

It is apparent to one skilled in the art that various changes and modifications can be made to this disclosure, and equivalents employed, without departing from the spirit and scope of the invention. For example, the piezoelectric driver 1325 and the drive element 1330 may take the form of any suitable mechanical coupling to translate the piezoelectric movement of the piezoelectric element 1320 into appropriate movement of the sphincter band 1310. In one alternative embodiment, the drive element 1330 may be an elongate body that is notched along its longitudinal axis. The notches may be in the form of a saw tooth or any other suitable shape, size or pitch to produce the desired degree of movement. The piezoelectric driver 1325 has an end configured to engage with the notches. Operation of the piezoelectric element 1320 in this alternative produces longitudinal movement of the notched drive element 1330 toward and/or away from the piezo motor housing 1315. As before, the sphincter band 1310 is attached to the housing 1315 and the drive element 1330 such that operation of the piezoelectric element 1320 produces the desired movement of the sphincter band 1310. Additionally, elements shown and described with any embodiment are exemplary for the specific embodiment and can be used on other embodiments within this disclosure.

What is claimed is:

1. An artificial sphincter comprising:
a piezoelectric element attached to a housing;
a drive element movably coupled to the housing;
a driver attached to the piezoelectric element and configured to translate a piezoelectric oscillation of the piezoelectric element into movement of the drive element; and
a sphincter band attached to the housing and configured to be driven by the drive element.

2. The artificial sphincter of claim 1 wherein the sphincter band has a length configured to encircle a body cavity, organ or lumen.

3. The artificial sphincter of claim 1, wherein the driver is configured to translate the piezoelectric oscillation of the piezoelectric element into longitudinal movement of the drive element.

4. The artificial sphincter of claim 1, wherein the driver is configured to translate the piezoelectric oscillation of the piezoelectric element into rotational movement of the drive element.

5. The artificial sphincter of claim 1, further comprising a power supply in electrical communication with the piezoelectric element.

6. The artificial sphincter of claim 1 wherein application of electrical current to said piezoelectric element increases or decreases a circumference formed by the sphincter band.

7. The artificial sphincter of claim 6 wherein the perimeter formed by the sphincter band is adapted and configured to at least partially encircle a body cavity.

8. The artificial sphincter of claim 1 further comprising a bellows between the drive element and the housing.

9. The artificial sphincter of claim 1 further comprising an inductive coupling mechanism adapted to connect the piezoelectric element to a power source.

10. The artificial sphincter of claim 7 wherein said body cavity is a urethra, lower esophagus, lower gastro-intestinal tract, or rectum.

11. A method of controlling passage of contents across a body cavity comprising:
implanting a control device around a body cavity, said device comprising an piezoelectric actuator, a sphincter band, a drive element connected between the piezoelectric actuator and the sphincter band, and a power management device;
controlling a flow of contents in said body cavity, said control being performed by constricting and unconstricting said body cavity by operating the piezoelectric actuator to decrease and increase the circumference of the sphincter band.

12. A method of treating a disease using an artificial sphincter comprising:
implanting a sphincter band around a body cavity, said sphincter band connected to a piezoelectric element; and
closing said body cavity with the sphincter band by converting movement of the piezoelectric element to movement to decrease a diameter of the sphincter band.

13. An artificial sphincter system comprising:
a sphincter band configured to be implanted around a body lumen;
a piezo motor coupled to the sphincter band such that the motor can cause the band to alternately constrict and release the body lumen;
an internal communications system configured to be implanted subcutaneously in electrical communication with the piezo motor; and
an external control system having a transmitter configured to send signals to the internal communications system for driving the piezo motor to constrict or release the sphincter band around the body lumen.

14. The artificial sphincter system of claim 13 wherein the external control system comprises a handheld unit.

15. The artificial sphincter system of claim 13 wherein the external control system provides power to the piezo motor.

16. The artificial sphincter system of claim 13 wherein the internal communications system comprises a power source.

17. The artificial sphincter system of claim 13 wherein body lumen is a urethra, lower esophagus, lower gastro-intestinal tract, or rectum.
